(19) United States
(12) Patent Application Publication
(10) Pub. No.: US 2024/0263081 A1
(43) Pub. Date: Aug. 8, 2024

(54) BIOMETHANE AND/OR HYDROGEN PRODUCED FROM BIOMASS HAVING REDUCED LIFECYCLE GREENHOUSE GAS EMISSIONS

(71) Applicant: Iogen Corporation, Ottawa (CA)

(72) Inventors: Brian Foody, Ottawa (CA); Patrick J. Foody, Ottawa (CA)

(21) Appl. No.: 18/600,023

(22) Filed: Mar. 8, 2024

Related U.S. Application Data
(63) Continuation of application No. PCT/CA2022/051769, filed on Dec. 2, 2022.
(60) Provisional application No. 63/379,945, filed on Oct. 18, 2022.
(60) Provisional application No. 63/368,812, filed on Jul. 19, 2022.
(60) Provisional application No. 63/264,923, filed on Dec. 3, 2021.

(51) Int. Cl. C10G 2/00; B01D 53/00; B01D 53/02; B01D 53/14; B01D 53/22; C01B 3/38; C10G 3/00; C10G 45/00; C10L 1/08; C12P 5/02

(52) U.S. Cl. CPC C10G 2/30; C01B 3/384; C10G 3/52; C10G 45/00; C10L 1/08; C12P 5/023; B01D 53/002; B01D 53/02; B01D 53/1475; B01D 53/22; B01D 2257/504; B01D 2258/0283; B01D 2258/05; C01B 2203/0233; C01B 2203/042; C01B 2203/0475; C10G 2300/4043; C10G 2300/42; C10G 2400/08; C10L 2270/04

(57) ABSTRACT

A process and/or system for producing biomethane, hydrogen, or fuel, fuel intermediate, and/or chemical product from the biomethane or hydrogen. The biomethane and/or hydrogen is produced in a process that converts biomass to biomethane. In certain embodiments, the biomethane production process includes anaerobic digestion, which produces biogas and digestate. Carbon-containing material (e.g., derived from the biomass) is stored and/or used as part of at least one carbon capture and storage process, where the carbon-containing material includes (i) carbon dioxide produced from the biomethane production process (e.g., produced from anaerobic digestion), and (ii) carbon-containing material obtained or derived from residue of the biomethane production process, and optionally includes (iii) carbon dioxide produced from the hydrogen production process.

### TECHNICAL FIELD

The present disclosure relates to a process and/or system for converting biomass to biomethane, which can be used to produce hydrogen, where the biomethane and/or hydrogen has reduced lifecycle greenhouse gas (GHG) emissions at least in part because carbon from residue of the biomethane production is stored/used as part of at least one carbon capture and storage process. The present disclosure also relates to a process and/or system that produces fuel, fuel intermediate, and/or chemical product from such biomethane and/or hydrogen.

### BACKGROUND

Fossil fuels like coal, oil, and natural gas supply a large percentage of the world's energy. Fossil fuels are also the primary human source of greenhouse gas (GHG) emissions. Natural gas, which may account for nearly a quarter of GHG emissions, is used, for example, for power generation, for domestic use (e.g., cooking and heating), for transportation, and/or as feedstock (e.g., for producing ammonia, fertilizers, hydrogen, steel, plastics, etc.).

Hydrogen production is a significant consumer of natural gas and source of GHG emissions. For example, the production of hydrogen from the steam methane reforming (SMR) of natural gas, which is often referred to as grey hydrogen, contributes to high GHG emissions associated with oil refining and/or ammonia production. The GHG emissions from grey hydrogen production can be reduced by capturing and storing carbon dioxide (CO₂) produced from the SMR such that it is prevented from being released to the atmosphere (e.g., stored underground in suitable geological formations). Such carbon capture and storage (CCS), when combined with the SMR of natural gas, produces what is often referred to as blue hydrogen. Blue hydrogen can have a carbon intensity (CI) that is less than half that of grey hydrogen. However, blue hydrogen can still have significant lifecycle GHG emissions (e.g., fugitive methane emissions and/or emissions associated with the CCS process). Moreover, blue hydrogen is still reliant on fossil fuels. Concerns over climate change have imposed the need to reduce GHG emissions and/or reliance on fossil fuels.

One approach to reduce GHG emissions and/or reliance on fossil fuels is to use biomethane in place of natural gas, thereby avoiding GHG emissions associated with the use of the natural gas. However, the use of biomethane as a replacement for natural gas has been criticized in terms of its availability (e.g., the supply may be limited and/or inadequate to meet current natural gas demand), cost (e.g., often more than twice the cost of natural gas), and carbon intensity. With regard to the latter, the carbon intensity of biomethane may, for example, be dependent on GHG emissions associated with providing heat for the anerobic digestion, electricity used for biogas upgrading and/or compressing the biomethane (e.g., prior to injection into a natural gas distribution system), and/or methane leakage (e.g., from open lagoons and/or from fugitive methane emissions). For example, when biomethane is transported using a natural gas distribution system it can be associated with the same type of pipeline emissions associated with natural gas. The significant GHG emissions associated with biomethane production may be accentuated when the biomethane is used in a process also having significant GHG emissions. For example, when biomethane is used in place of natural gas in hydrogen production, the carbon intensity of hydrogen produced using certain biomethanes can be greater than that of blue hydrogen.

### SUMMARY

The present disclosure relates generally to process(es) and/or system(s) that may help mitigate or obviate one or more of the alleged disadvantages of using biomethane in place of natural gas to decrease global GHG emissions. For example, the present disclosure provides process(es)/system(s) where GHG emissions associated with using biomethane are reduced by capturing and storing at least: (i) carbon dioxide produced from the biomethane production process (e.g., carbon dioxide from biogas produced from anaerobic digestion), and (ii) carbon-containing material obtained and/or derived from residue of the biomethane production process (e.g., part of the biomass not converted to biogas).

Advantageously, the residue from biomethane production typically contains carbon that was recently fixed by photosynthesis, and storing it can prevent or delay its release to the environment. The corresponding reduction in GHG emissions can be particularly significant when the biomethane production is based on anaerobic digestion and the residue is digestate. The anaerobic digestion of many feedstocks (e.g., fibrous biomass and/or manure) is often incomplete. For example, while a large portion of the carbon (e.g., about 50%, by mass) from the biomass is often converted to biogas (e.g., to carbon dioxide and methane), a large portion (e.g., about 50%, by mass) may remain in the digestate (e.g., in unconverted lignin, cellulose, etc.). Accordingly, processing the digestate so as to be able to capture and store at least some of this carbon can significantly reduce lifecycle GHG emissions of the biomethane, hydrogen produced from the biomethane, or fuel, fuel intermediate, or chemical product, produced from the biomethane and/or hydrogen. In addition, the reduction in GHG emissions (i.e., relative to not capturing carbon originating from the digestate) can be increased when the processing of the digestate also produces heat and/or power that can be used within the process (e.g., the biomethane production process).

In accordance with one aspect of the instant invention there is provided a process for producing fuel, fuel intermediate, chemical product, or any combination thereof, the process comprising: providing biomethane, the biomethane produced from a biomethane production process comprising converting biomass to biomethane, wherein the biomethane is used to generate hydrogen in a hydrogen production process, the hydrogen production process comprising (a) providing methane-containing feedstock comprising the biomethane, (b) subjecting at least part of the methane-containing feedstock to methane reforming, thereby producing syngas, the methane reforming conducted in one or more reactors, and, (c) subjecting the syngas to a purification process wherein hydrogen is separated from at least carbon dioxide, thereby producing a stream enriched in hydrogen, wherein carbon-containing material is stored and/or used as part of at least one carbon capture and storage process, the carbon-containing material comprising (i) carbon dioxide produced from the biomethane production process, (ii) carbon dioxide produced from the hydrogen production process, and (iii) residue from the biomethane production process, or carbon-containing material derived from the residue.

In accordance with one aspect of the instant invention there is provided a process for producing biomethane comprising: subjecting biomass to anaerobic digestion, thereby producing biogas and digestate, the biogas comprising methane and carbon dioxide; subjecting the biogas to biogas upgrading, thereby removing at least some of the carbon dioxide from the biogas and producing upgraded biogas; providing at least some of the removed carbon dioxide as part of a first carbon capture and storage process; combusting at least part of the digestate, thereby producing flue gas comprising carbon dioxide; and capturing at least some of the carbon dioxide from the flue gas and providing the captured carbon dioxide as part of the first carbon capture and storage process, a second other carbon capture and storage process, or a combination thereof.

In accordance with one aspect of the instant invention there is provided a process for producing biomethane comprising: providing feedstock comprising fibrous biomass; reducing an average particle size of the fibrous biomass; subjecting the fibrous biomass having a reduced average particle size to anaerobic digestion, thereby producing biogas and digestate, the biogas comprising methane and carbon dioxide; subjecting the biogas to biogas upgrading, thereby removing at least some of the carbon dioxide from the biogas, and producing upgraded biogas; providing at least some of the removed carbon dioxide as part of a first carbon capture and storage process; subjecting at least part of the digestate to a liquid solid separation, thereby producing a solids stream and a liquid stream; combusting at least part of the solids stream and optionally at least part of the liquid stream, thereby producing flue gas comprising carbon dioxide; and capturing at least some of the carbon dioxide from the flue gas and providing the captured carbon dioxide as part of the first carbon capture and storage process, a second other carbon capture and storage process, or a combination thereof.

In accordance with one aspect of the instant invention there is provided a process for producing ammonia comprising: providing biomethane, the biomethane produced in a process comprising: (i) subjecting biomass to anaerobic digestion, thereby producing biogas and digestate, the biogas comprising methane and carbon dioxide; (ii) subjecting the biogas to biogas upgrading, thereby removing at least some of the carbon dioxide from the biogas, and producing the biomethane; and (iii) subjecting at least part of the digestate to combustion, thereby producing flue gas comprising carbon dioxide, wherein at least some of the carbon dioxide removed from the biogas and at least some of carbon dioxide removed from the flue gas is provided as part of at least one carbon capture and storage process; producing hydrogen using the biomethane in a hydrogen production process that includes carbon capture and storage; using the hydrogen in ammonia production.

In accordance with one aspect of the instant invention there is provided a process for producing fuel, fuel intermediate, chemical product, or any combination thereof, the process comprising: providing biomethane, the biomethane produced from a biomethane production process comprising subjecting biomass to anaerobic digestion, the anaerobic digestion generating digestate and biogas, the biogas comprising methane and carbon dioxide; and using the biomethane in a hydrogen production process comprising methane reforming and hydrogen purification, the methane reforming conducted in one or more reactors and producing syngas comprising hydrogen and carbon dioxide, at least part of the syngas subjected to the hydrogen purification, thereby producing a stream enriched in hydrogen, wherein carbon-containing material derived from the biomass and not converted to biomethane is stored and/or used as part of at least one carbon capture and storage process, the carbon-containing material comprising (i) at least a portion of the carbon dioxide generated from the anaerobic digestion, and (ii) carbon-containing material derived from the digestate.

### BRIEF DESCRIPTION OF THE DRAWINGS

Further features and advantages of the present disclosure will become apparent from the following detailed description, taken in combination with the appended drawings, in which:

FIG. 1a is a process flow diagram in block form in accordance with an embodiment of the instant disclosure;

FIG. 1b is a process flow diagram in block form in accordance with another embodiment of the instant disclosure;

FIG. 2 is a process flow diagram in block form in accordance with another embodiment of the instant disclosure;

FIG. 3 is a process flow diagram in block form in accordance with an embodiment of the instant disclosure, wherein the biomethane production comprises anaerobic digestion;

FIG. 4 is a process flow diagram in block form in accordance with an embodiment of the instant disclosure, wherein the biomethane production comprises multiple anaerobic digestions;

FIG. 5 is a process flow diagram in block form in accordance with an embodiment of the instant disclosure, wherein the biomethane production comprises gasification;

FIG. 6a is process flow diagram in block form of a hydrogen production process that includes SMR wherein the heat for reforming is provided by combusting methane-containing gas;

FIG. 6b is a process flow diagram in block form of a hydrogen production process that includes SMR wherein the heat for reforming is provided by combusting methane-containing fuel gas and off gas from hydrogen purification; and

FIG. 7 is a schematic diagram for producing ammonia according to an embodiment of the invention.

It will be noted that throughout the appended drawings, like features are identified by like reference numerals

### DETAILED DESCRIPTION

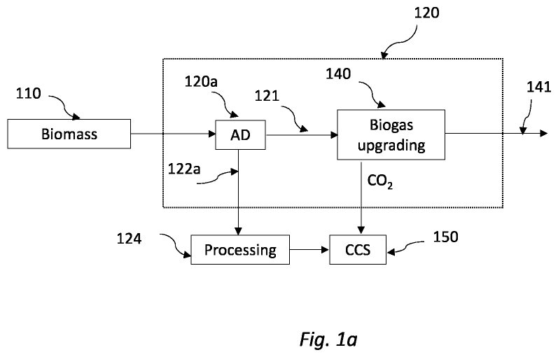

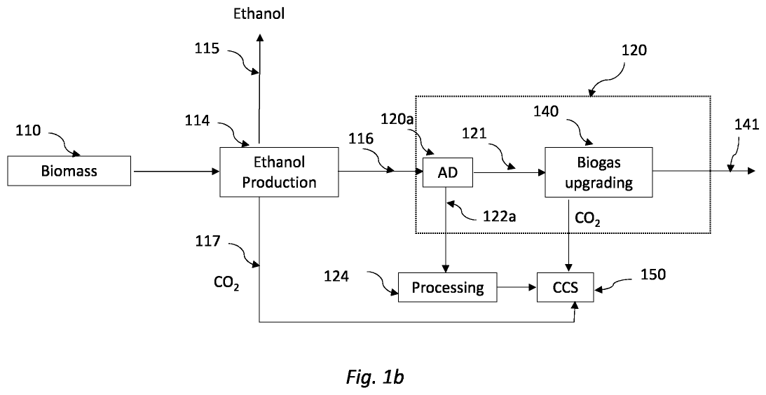

Referring to FIGS. 1 through 5, there is shown illustrative process(es) and/or system(s) according to certain embodiments of the instant disclosure. Prior to describing these figures in more detail, it is to be noted that the figures are simplified flow diagrams and do not necessarily reflect all of the steps/units/equipment that may be incorporated. The incorporation of such steps/units/equipment is well known and will be understood by those skilled in the art.

### Biomass

The process(es) and/or system(s) of the instant disclosure produce and/or use biomethane derived from biomass 110. Biomass refers to organic material originating from plants, animals, or micro-organisms (e.g., including plants, agricultural crops or residues, municipal wastes, animal wastes, and algae). Biomass is a renewable resource, which can be naturally replenished on a human timescale, and which can be used to produce bioenergy and/or biofuels (e.g., biogas). In general, the biomass 110 can be any suitable biomass (e.g., one or more types of biomass feedstock). Some examples of suitable biomass may include: (i) energy crops (e.g., switchgrass, sorghum, etc.); (ii) residues, byproducts, or waste from the processing of plant material in a facility, or feedstock derived therefrom (e.g., sugarcane bagasse, sugarcane tops/leaves, corn stover, etc.); (iii) agricultural residues (e.g., wheat straw, corn cobs, barley straw, corn stover, etc.); (iv) forestry material; (v) livestock manure, including sheep, swine, and cow manure; (vi) food scraps and/or agrifood processing residues (e.g., from slaughterhouse), and/or (vii) municipal waste or components removed or derived from municipal waste. These examples of suitable biomass are advantageous in that they do not compete with food production. The use of forestry or agricultural feedstocks (e.g., energy crops, residues, byproducts, or waste from the processing of plant material in a facility, or feedstock derived therefrom, or agricultural residues) may be advantageous for reducing GHG emissions. The use of livestock manure, such as swine or cow manure, may be advantageous in terms of reducing the lifecycle GHG emissions the hydrogen, or fuel, fuel intermediate, or product (e.g., chemical product) produced using the hydrogen. The use of fibrous biomass (e.g., bagasse, coconut husk, straw, reed, alfalfa, etc.) and/or biomass having fibrous component, may be advantageous in terms of having the potential to increase the supply of biogas. For example, while the supply of biogas from landfills and/or manure is substantially limited, the use of fibrous biomass to produce biogas has the potential to increase supply. Advantageously, the supply can be increased using agricultural residues.

### Biomethane Production

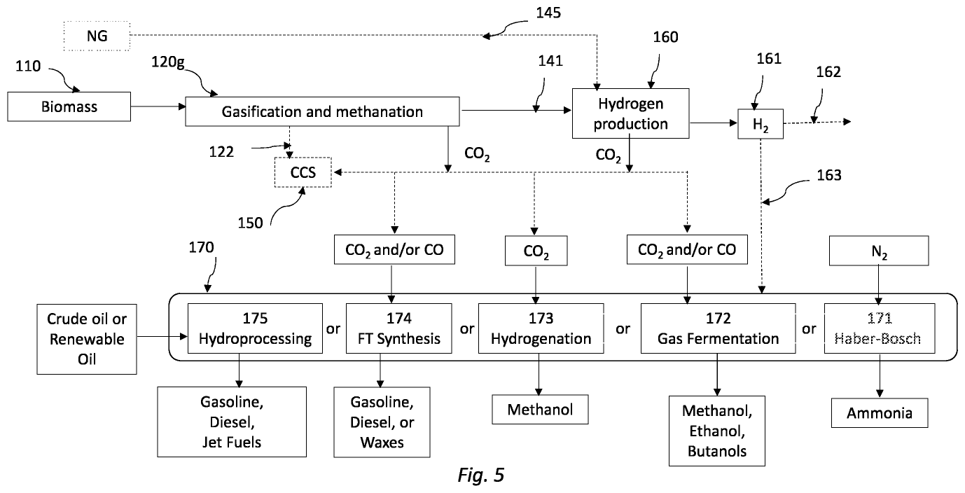

In general, at least part of the biomass is converted to upgraded biogas (e.g., biomethane) in biomethane production 120. Biomethane production can include any suitable process or combination of processes that can convert at least part of the biomass to upgraded biogas (e.g., biomethane). For example, the biomethane production process can include anaerobic digestion 120a, which produces biogas, and biogas upgrading 140 as illustrated in FIGS. 1a, 1b, 3, and 4. The term “biogas”, as used herein, refers to a gas mixture that contains methane produced from biomass. Alternatively, the biomethane production process can include gasification and methanation 120g as illustrated in FIG. 5, or pyrolysis (not shown). In addition to producing upgraded biogas (e.g., biomethane) 141, the biomethane production may produce a residue (e.g., carbon-containing material 122/122a) that is not converted to biogas/biomethane. This residue, or a carbon-containing material produced by processing 124 (e.g., combusting) at least part of this residue 122/122a is provided and/or stored as part of one or more carbon-capture and storage processes. The phrase “derived from biomass”, with reference to upgraded biogas, biomethane, or carbon-containing material, means that the upgraded biogas, biomethane, or carbon-containing material, respectively, is produced from the biomass from one or more processes (e.g., directly or indirectly). The phrase “derived from residue”, with reference to carbon-containing material, means that the carbon-containing material is produced from the residue (e.g., liquid and/or solid) from one or more processes (e.g., directly or indirectly).

Referring to the embodiments in FIG. 1a, 1b, 3, and 4, at least part of the biomass is subjected to anaerobic digestion 120a. Anaerobic digestion refers to the biological breakdown of organic matter by anacrobic microorganisms, is typically conducted in anaerobic or low oxygen conditions, and may involve a series of microorganism types and processes (e.g., hydrolysis, acidogenesis, acetogenesis, and methanogenesis). In general, the anaerobic digestion of biomass can be conducted in any suitable environment, including a natural environment (e.g., a landfill) or a controlled environment (e.g., one or more anaerobic digesters arranged in series and/or in parallel). For purposes herein, the anaerobic digestion 120a is conducted in one or more anaerobic digesters (e.g., in series and/or in parallel). Each anaerobic digester can be a holding tank, or another contained volume, such as a covered lagoon or sealed structure, configured to facilitate the anaerobic digestion and collection of biogas. For example, each anaerobic digester can be a plug flow system or basin type reactor. Such anaerobic digesters can be single-stage or multi-stage digester systems and/or may be designed and/or operated in a number of configurations including batch or continuous, mesophilic or thermophilic temperature ranges, mixed or unmixed, and low, medium, or high rates. The anaerobic digestion conducted in such digesters can use a nutrient solution, which may improve the conversion, particularly for fibrous biomass. Using a controlled environment facilitates monitoring input and output material flows, which can be used to determine how much biogas is produced from the anaerobic digestion of a certain amount of biomass, and/or which can be used to calculate lifecycle GHG emissions and/or validate compliance (e.g., with a pathway).

In general, the feedstock for anaerobic digestion 120a can be any suitable biomass. For example, it can be raw or pretreated biomass, or can be biomass that is produced from another process (e.g., can be waste, residue, and/or byproduct from another process).

Referring to FIG. 1a, the biomass fed to anaerobic digestion is raw or pretreated biomass. The biomass may be subjected to anaerobic digestion as substantially intact bales or may be unbaled prior to anaerobic digestion. The biomass (e.g., baled or unbaled) may be pretreated prior to the anaerobic digestion. Such pretreatment can include size reduction, sand removal, slurry formation, the addition of chemicals and/or heat (e.g., steam explosion), and/or nutrients provided for the anaerobic digestion. Advantageously, size reduction can accelerate the anaerobic digestion process and/or improve material handling. Some examples of size reduction methods include milling, grinding, agitation, shredding, compression/expansion, and/or other types of mechanical action. Size reduction by mechanical action may be performed by any type of equipment adapted for the purpose, for example, but not limited to, hammer mills, tub-grinders, roll presses, refiners, hydropulpers, and hydrapulpers. In certain embodiments, biomass having an average particle size that is greater than about 6-8 inches is subject to a size reduction wherein at least 90% by volume of the particles produced from the size reduction have a length between about 1/16 inch and about 6 inches.

Referring to FIG. 1b, the biomass fed to anaerobic digestion includes waste and/or residue 116 from ethanol production 114. Such ethanol production 114, may for example, produce corn ethanol, sugar ethanol, or cellulosic ethanol 115, in addition to carbon dioxide 117. The waste and/or residue 116 can include aqueous streams (e.g., condensate streams), solids filtered from one or more streams (e.g., after hydrolysis), and/or at least part of the still bottoms (e.g., from ethanol recovery). In certain embodiments, carbon dioxide 117 from ethanol production is provided as part of one or more carbon capture and storage processes 150.

The biogas 121 produced by the anaerobic digestion of biomass is a gas mixture that typically contains methane (CH₄) and carbon dioxide (CO₂), and that may contain water (H₂O), nitrogen (N₂), hydrogen sulfide (H₂S), ammonia (NH₃), oxygen (O₂), volatile organic compounds (VOCs), and/or siloxanes, depending on the biomass from which it is produced. Biogas produced from anaerobic digestion often has a methane content between about 35% and 75% (e.g., about 60%) and a carbon dioxide content between about 15% and 65% (e.g., about 35%). The percentages used to quantify gas composition and/or a specific gas content, as used herein, are expressed as mol %, unless otherwise specified. More specifically, they are expressed by mole fraction at standard temperature and pressure (STP), which is equivalent to volume fraction.

When conducted in one or more anaerobic digesters, the anaerobic digestion of biomass also produces a potentially usable digestate 122a. Digestate refers to the liquid and/or solid material remaining after one or more stages of anaerobic digestion (e.g., may refer to acidogenic digestate, methanogenic digestate, or a combination thereof). Digestate can include organic material not digested by the anaerobic microorganisms (e.g., fibrous undigested organic material made of lignin and cellulose), by-products of the anaerobic digestion released by the microorganisms, and/or the microorganisms themselves. For example, the digestate can include carbohydrates, nutrients (such as nitrogen compounds and phosphates), other organics, and/or wild yeasts. The composition of digestate can vary depending on the biomass from which it is derived. Digestate often has both a solid and liquid component. One use of digestate is as a soil conditioner, where it can provide nutrients for plant growth and/or displace the use of fossil-based fertilizers. However, as a soil conditioner, digestate may have a significant methane formation potential, and thus may be associated with GHG emissions. In certain embodiments of the instant disclosure, the digestate 122a is processed 124 (e.g., combusted) to provide carbon-containing material that that is stored as part of CCS.

The biogas produced in anaerobic digestion 120a is subjected to biogas upgrading 140. Biogas upgrading refers to a process where biogas (e.g., raw or cleaned biogas) is treated to remove one or more components (e.g., CO₂, N₂, H₂O, H₂S, O₂, NH₃, VOCs, siloxanes, and/or particulates), wherein the treatment increases the calorific value of the biogas. For example, biogas upgrading typically includes removing carbon dioxide and/or nitrogen. In general, biogas upgrading can be conducted using any suitable technology or combination of technologies known in the art. Biogas upgrading, which is well-known, often includes one or more of the following technologies: 1) absorption, 2) adsorption, 3) membrane separations, and 4) cryogenic upgrading. As will be understood by those skilled in the art, the technology or combination of technologies selected may be dependent on the composition of the biogas and/or how it is produced. Since biogas often has a significant carbon dioxide content, biogas upgrading plants often include at least one system for separating methane from carbon dioxide. Some examples of technologies that can remove carbon dioxide from biogas include, but are not limited to, absorption (e.g., water scrubbing, organic physical scrubbing, chemical scrubbing (e.g., amine)), adsorption (e.g., pressure swing adsorption (PSA), which includes vacuum PSA, or temperature swing adsorption), membrane separation (e.g., CO₂ selective membranes based on polyimide, polysulfone, cellulose acetate, polydimethylsiloxane), and cryogenic separation. Optionally, biogas upgrading can include increasing the calorific value of the biogas by adding gas having a relatively high energy content (e.g., propane, natural gas).

Preferably, the biogas upgrading 140 produces biomethane. When produced from biogas upgrading, biomethane refers to: (1) biogas that has been upgraded to meet or exceed applicable natural gas distribution system specifications (e.g., pipeline specifications), (2) biogas that has been upgraded to meet or exceed applicable quality specifications for vehicle use (e.g., CNG specifications), and/or (3) natural gas withdrawn from a natural gas distribution system that is associated with the environmental attributes of upgraded biogas injected into the natural gas distribution system (e.g., a gas that qualifies as biomethane under applicable regulations). With respect to (1), pipeline specifications, which can include specifications required for biogas for injection into a natural gas distribution system, may vary by region and/or country in terms of value and units. For example, pipelines standards may require the biomethane to have a CH₄ content that is at least 95% or have a heating value of at least 950 BTU/scf. With respect to (3), since the transfer or allocation of the environmental attributes of the upgraded biogas injected into the natural gas distribution system to gas withdrawn at a different location is typically recognized, the withdrawn gas is recognized as biomethane and/or qualifies as biomethane under applicable regulations (e.g., even though the withdrawn gas may not contain actual molecules from the original biomass and/or contains methane from fossil sources). Such transfer may be carried out on a displacement basis, where transactions within the natural gas distribution system involve a matching and balancing of inputs and outputs. Typically, the direction of the physical flow of gas is not considered. The term “environmental attributes”, as used herein with regard to a specific material (e.g., biomethane), refers to any and all attributes related to the material, including all rights, credits, benefits, or payments associated with the renewable nature of the material and/or the reduction in or avoidance of fossil fuel consumption or reduction in lifecycle GHG gas emissions associated with the use of the material. Some non-limiting examples of environmental attributes include verified emission reductions, voluntary emission reductions, offsets, allowances, credits, avoided compliance costs, emission rights and authorizations, certificates, voluntary carbon units, under any law or regulation, or any emission reduction registry, trading system, or reporting or reduction program for GHG gas emissions that is established, certified, maintained, or recognized by any international, governmental, or nongovernmental agency.

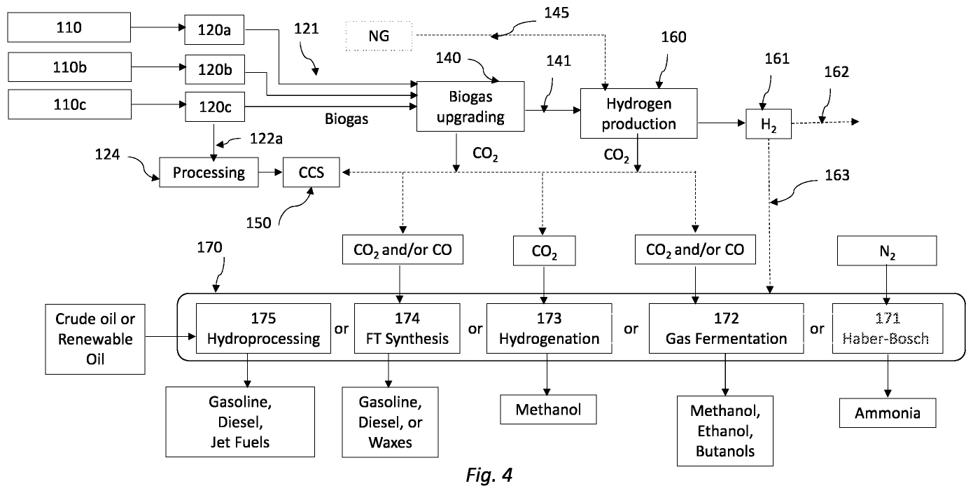

In general, the biogas upgrading 140 can be conducted at one or more biogas upgrading facilities. For example, in certain embodiments the biogas 121 provided for biogas upgrading includes multiple biogases, where each biogas is produced from a different anaerobic digestion 120a, 120b, 120c, which can have different biomass feedstocks 110, 110b, 110c, as illustrated in FIG. 4. In this case, the biogas upgrading 140 can be conducted at a plurality of decentralized biogas upgrading facilities (not shown), each biogas upgrading facility being in close proximity to one of the anaerobic digestions 120a, 120b, 120c. Alternatively, or additionally, the biogas upgrading can be conducted at a centralized biogas upgrading facility that receives raw or partially purified biogases produced from the different anaerobic digestions 120a, 120b, 120c. When the biogas upgrading 140 produces biomethane 141, the biomethane produced can be transported using a natural gas distribution system (if required). Accordingly, the biomethane can be transported cost effectively and/or from a relatively large geographical area. Providing biomethane produced from biomass from different sources (e.g., different farms), and in particular from different biomethane producers, can be advantageous for providing biomethane having a carbon intensity below a certain value and/or to increase scale of the process.

Referring to the embodiment in FIG. 5, the biomass is subjected to gasification followed by methanation 120g. Gasification refers to a process that converts biomass and/or fossil-based carbonaceous materials at high temperatures (e.g., >700° C.), without combustion, with a controlled amount of oxygen and/or steam into gas mixture primarily composed of carbon monoxide (CO) and hydrogen and sometimes carbon dioxide, referred to as syngas. For example, the syngas produced by the gasification of wood may include carbon monoxide, carbon dioxide, hydrogen, methane, ethylene (C₂H₄), ethane (C₂H₆), dust (ash), tar, chloride, sulfur, etc. Following gasification, the syngas is often subjected to cooling, tar removal, and/or cleaning. The syngas may then be subjected to methanation, a catalytic conversion wherein carbon dioxide and carbon monoxide in the syngas can undergo the following reactions:

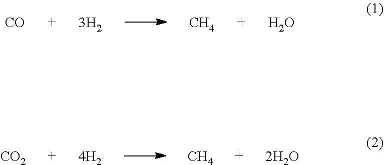

Methanation, which is well-known in the art, typically is carried out in the presence of a solid catalysis (e.g., nickel-based catalyst). The gas produced by this gasification and methanation approach typically contains methane (and possibly ethane) and water, and can include carbon dioxide. The gas can be purified and/or dried to provide biomethane. Methanation units, which can include a water gas shift reactor, a carbon dioxide scrubber, a methanation reactor, and a dehydration system, are often configured to produce biomethane. When produced from gasification of biomass followed by methanation, biomethane refers to: (1) a near-pure source of methane derived from the biomass that can meet or exceed applicable natural gas distribution system specifications (e.g., pipeline specifications), (2) a near-pure source of methane derived from the biomass that can meet or exceed applicable quality specifications for vehicle use (e.g., CNG specifications), and/or (3) natural gas withdrawn from a natural gas distribution system that is associated with the environmental attributes of a near-pure source of methane derived from the biomass and injected into the natural gas distribution system (e.g., a gas that qualifies as biomethane under applicable regulations). A possible byproduct of biomass gasification is biochar (biological charcoal). Carbon-containing material not converted to biomethane 122 (e.g., residue such as biochar) and/or carbon dioxide produced from gasification may be provided for use as part of CCS.

### Hydrogen Production

In certain embodiments, the biomethane 141 is used in hydrogen production 160. In general, the hydrogen production can use any suitable technology known in the art that can convert methane-containing gas such as biomethane and/or natural gas to hydrogen. Examples of technologies that may be suitable include, but are not limited to, steam methane reforming (SMR), autothermal reforming (ATR), partial oxidation (POX), and dry methane reforming (DMR). SMR, ATR, and DMR, which are types of catalytic reforming, may operate by exposing natural gas to a catalyst at high temperature and pressure to produce syngas. POX reactions, which include thermal partial oxidation reactions (TPOX) and catalytic partial oxidation reactions (CPOX), may occur when a sub-stoichiometric fuel-oxygen mixture is partially combusted in a reformer. POX also may be referred to as oxidative reforming. For purposes herein, the term “methane reforming” may refer to SMR, ATR, DMR, or POX. Methane reforming is well known in art. Of the various types of methane reforming, SMR is the most common.

In certain embodiments, the hydrogen production includes SMR. In SMR, which is an endothermic process, methane is reacted with steam under pressure in the presence of a catalyst to produce carbon monoxide (CO) and H2 according to the following reaction:

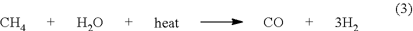

The SMR reaction may occur in the SMR reactor tubes, which contain the reforming catalyst. Without being limiting, the catalyst may be nickel-based, the operating pressure may be between 200 psig (1.38 MPa) and 600 psig (4.14 MPa), and the operating temperature may be between about 450 to 1000° C.

In certain embodiments, the hydrogen production includes DMR. In DMR, methane reacts with carbon dioxide, rather than water, according to the following reaction:

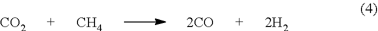

Without being limiting, the DMR catalyst may be nickel, iron, ruthenium, palladium, or platinum based. While the DMR process does not require steam, and may be conducted at lower temperatures, it may be limited by the potential for coke formation.

In certain embodiments, the hydrogen production includes ATR. ATR combines partial oxidation and catalytic steam or carbon dioxide reforming of methane in a single reactor. Heat generated from the partial oxidation (e.g., in the combustion zone of the reactor) may be used in the catalytic reforming (e.g., in the reforming zone of the reactor). Accordingly, a common stand-alone ATR may not require the supply or dissipation of thermal energy. The ATR reactions include:

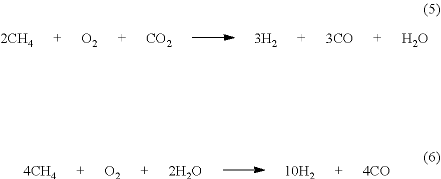

The syngas produced from methane reforming (e.g., Eqs. 3, 4, 5, or 6) may be further reacted in a water gas shift (WGS) reaction, wherein carbon monoxide is converted to carbon dioxide and hydrogen:

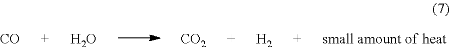

Although optional, providing WGS downstream of methane reforming increases the yield of H₂, and thus is commonly included in hydrogen production. When included, the WGS is considered to be part of the methane reforming. The syngas produced from methane reforming often includes hydrogen, methane, carbon monoxide, carbon dioxide and water vapour. As will be understood by those skilled in the art, methane reforming can be conducted using one or more reactors. For example, the WGS can be conducted using a high temperature WGS reactor followed by a low temperature WGS reactor.

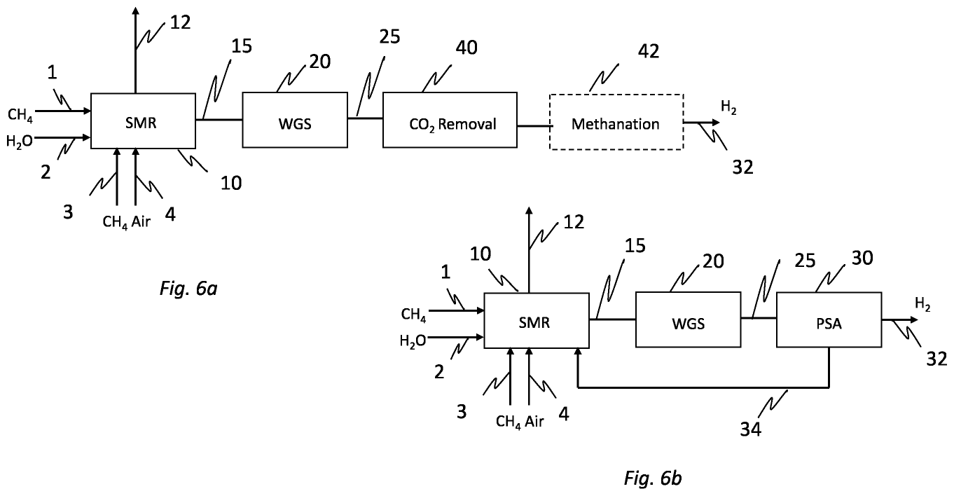

When the hydrogen production includes methane reforming (e.g., SMR) where heat is required for the catalytic reforming, the heat can be generated using low-carbon electricity and/or by combusting methane-containing gas (e.g., biomethane). In certain embodiments, at least part of the heat required for the catalytic reforming is provided by combusting methane-containing gas in the reformer burners (e.g., a combustion chamber may surround the reformer tubes that contains the catalyst and in which the reforming reaction is conducted). For example, consider the SMR illustrated in FIGS. 6a and 6b. Methane-containing feedstock is provided for hydrogen production. A portion of this feedstock is preheated and is fed as feed stream 1, along with steam 2, into the reactor tubes for the methane reforming 10, which contain the reforming catalyst. Another portion is provided as fuel gas 3, which is fed along with combustion air 4 into the reformer burners, which provide heat (e.g., required for an endothermic reforming reaction). The reformers may be characterized by the location of the burners within the combustion chamber (e.g., side-fired, top-fired, bottom-fired). Such fired burners are commonly used in hydrogen production. The syngas 15 produced from the methane reforming may be fed to WGS 20 to produce more hydrogen.

In addition to methane reforming, the hydrogen production includes a hydrogen purification process. In the hydrogen purification process, the syngas 15/25 produced from methane reforming (e.g., following WGS) is subjected to processing wherein hydrogen is separated from carbon monoxide, carbon dioxide, and/or methane in one or more stages to produce a stream enriched in hydrogen (i.e., containing at least 80% hydrogen). For example, in one embodiment, the hydrogen purification produces an enriched hydrogen stream having a hydrogen content of at least 90, 92, 94, 96, 98, 99, or 99.5%. In one embodiment, the hydrogen purification produces an enriched hydrogen stream having a hydrogen content of at least 99.9%. Without being limiting, some examples of suitable hydrogen purification technologies include, but are not limited to: a) absorption, b) adsorption, c) membrane separation, d) cryogenic separation, and/or e) methanation. Some examples of absorption systems that may be suitable include, but are not limited to, a monoethanolamine (MEA) unit or a methyldiethanolamine (MDEA) unit. A MEA unit may include one or more absorption columns containing an aqueous solution of MEA at about 30 wt %. The outlet liquid stream of solvent may be treated to regenerate the MEA and separate carbon dioxide. Some examples of adsorption systems that may be suitable include, but are not limited to, systems that use adsorbent bed (e.g., molecular sieves, activated carbon, active alumina, or silica gel) to remove impurities such as methane, carbon dioxide, carbon monoxide, nitrogen, and/or water from the syngas. Methanation is a catalytic process that can be conducted to convert the residual carbon monoxide and/or carbon dioxide in the syngas to methane. For example, see Eqs. 1 and 2. Since the methanation reaction consumes hydrogen, a hydrogen purification unit that includes a methanation may include carbon dioxide removal prior to methanation.

In general, the configuration of the hydrogen production process and/or plant can be dependent on the type of the methane reforming and/or hydrogen purification process provided. For example, consider the two hydrogen production processes based on SMR as illustrated in FIGS. 6a and 6b. In FIG. 6a, the syngas 25 produced from WGS 20, which may also be referred to as shifted gas, is cooled (not shown) and subjected to a hydrogen purification process that includes a wet scrubbing carbon dioxide removal process 40 (e.g., amine absorption and regeneration cycle), and optionally includes a methanation process 42 to convert any remaining carbon monoxide and/or carbon dioxide to methane. In FIG. 6b, the hydrogen purification process includes pressure swing adsorption (PSA) 30. The PSA 30 produces a stream enriched in hydrogen 32 and off gas 34. The off gas 34, which may contain unconverted methane, hydrogen, carbon dioxide, and/or carbon monoxide, is fed back to SMR 10, where it is used to provide additional process heat for the SMR (e.g., fuel the SMR burners). More specifically, the off gas 34 is combusted together with the fuel 3. The use of PSA, and more specifically, the recycle of the off gas to fuel the SMR burners, is generally associated with improved energy efficiency as less fuel gas 3 is required.

As discussed herein, the feedstock for hydrogen production preferably contains biomethane. It can be advantageous to use biomethane (e.g., relative to biogas that does not qualify as biomethane) because existing methane reformers may be configured to process natural gas and/or may operate more efficiently for biomethane and/or natural gas. For example, biogas that fails to qualify as biomethane may include impurities that poison the reforming catalysts. In addition, using biomethane facilitates providing the biomethane via a natural gas distribution system (e.g., the natural gas grid). In certain embodiments, the feedstock for hydrogen production also includes one or more other gases (e.g., non-renewable methane-containing gas such as fossil-based natural gas, refinery gas, liquid petroleum gas (LPG), light naphtha, etc.). Providing feedstock for hydrogen production that contains both biomethane and non-renewable methane-containing gas may provide scaling advantages for producing fuels, fuel intermediates, or products (e.g., chemical products) from biomass feedstock. When feedstock for hydrogen production includes both biomethane and non-renewable methane-containing gas, the biomethane can be allocated as either feed for the methane reforming and/or as fuel for providing heat for the methane reforming. The allocation can be conducted by physically directing it to either the reforming tube(s) or the burners, or using mass balance. In certain embodiments, the biomethane is allocated disproportionally between feed for the methane reforming and/or feed used as fuel for providing heat for the reforming (e.g., all of the biomethane provided for the methane reforming or all of the biomethane provided for fuel for providing heat for the reforming). In certain embodiments, all or at least some of the biomethane is subjected to methane reforming. In certain embodiments, all or at least some of the biomethane is used as fuel for the reforming.

The hydrogen production process 160 produces hydrogen 161. Advantageously, the hydrogen produced using upgraded biogas (e.g., biomethane) may be considered renewable hydrogen. Hydrogen, which can be used in gas or liquid form, is a very versatile as it can be used as a fuel, converted into electricity, and/or converted to one or more fuels, fuel intermediates, or chemical products. For example, renewable hydrogen can power fuel cell electric vehicles (FCEVs), which emit no tailpipe emissions other than water, can be run through a fuel cell to power the electricity grid, or used as rocket fuel.

In certain embodiments, the hydrogen is provided as a product 162 (e.g., for use in a fuel cell or a fuel). For example, the hydrogen can be used for transportation purposes, for generating electricity, and/or for use in district heating.

### Using the Hydrogen to Produce Fuel, Fuel Intermediate, Chemical Product

In certain embodiments, the hydrogen is provided as feedstock 163 in a production process that produces a fuel, fuel intermediate, chemical product, or any combination thereof. A fuel refers to a material (e.g., solid, liquid, or gaseous), which may contain carbon, that can be combusted to produce power and/or heat (e.g., may be transportation or heating fuel). A fuel intermediate is a precursor used to produce a fuel by a further conversion process, such as by a biologic conversion, a chemical conversion, or a combination thereof. A chemical product refers to a chemical compound used in a production process or a product such as a commodity. An example of a chemical product produced from hydrogen is fertilizer.

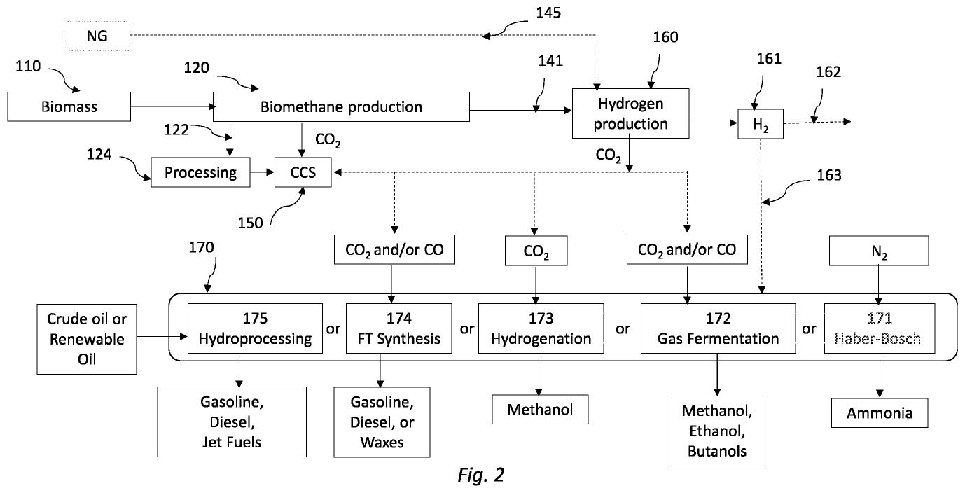

In certain embodiments, the hydrogen is provided as feedstock 163 to produce a fuel selected from long-haul trucking fuel (e.g., diesel), shipping fuel (e.g., heavy fuel oil), aviation fuel (e.g., kerosene, jet fuel) or district heating fuel. In certain embodiments, the hydrogen is provided as feedstock 163 to produce fuels or chemical products such as ammonia or fertilizer. Without being limiting some examples of suitable processing 170 are shown in FIGS. 2-5.

In certain embodiments, the hydrogen is used to produce ammonia in a Haber-Bosch process 171. In the Haber-Bosch process, which is well-known to those skilled in the art, nitrogen is converted to ammonia according to the following reaction:

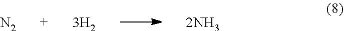

The reaction is conducted under high temperatures and pressures with a metal catalyst. Ammonia has an important role in the agricultural industry for production of fertilizers. Ammonia may also be used as an energy carrier for energy storage and transportation.

In certain embodiments, the hydrogen is used to produce one or more alcohols via gas fermentation 172. In gas fermentation, which is well-known to those skilled in the art, a gas mixture typically containing hydrogen with carbon dioxide and/or carbon monoxide is fed into a fermentation tank. In this embodiment, the carbon monoxide in the syngas functions as a substrate for the biologic conversion, which utilizes microorganisms or other biocatalysts. For example, acetogenic microorganisms can be used to produce a fermentation product from carbon monoxide. The production of ethanol by the acetogenic microorganisms proceeds through a series of biochemical reactions. Without being bound by any particular theory, the reactions carried out by the microorganism are as follows:

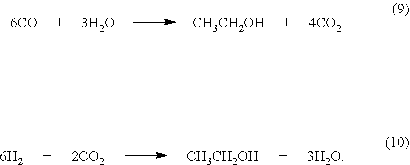

Some examples of strains that can produce ethanol from syngas are those from the genus Clostridium. In addition to ethanol, Clostridium bacteria may produce significant amounts of acetic acid (or acetate, depending on the pH) in addition to ethanol, depending upon process conditions. Such conditions can be readily selected by those of skill in the art and it should be appreciated that the invention is not constrained by any particular set of parameters selected for fermentation to improve productivity.

The fermentation products produced from gas fermentation, such as methanol, ethanol, or butanol, may be used as a fuel, or may be used to produce a fuel or chemical product. For example, ethanol may be used as a fuel directly or may be blended with gasoline. In addition, some technologies are able to convert various alcohols, including ethanol, into gasoline, diesel and jet fuel blendstocks, as well as produce benzene and/or toluene. In the embodiments including gas fermentation, the hydrogen may be used to supplement another gas feed containing carbon monoxide and/or carbon dioxide.

In certain embodiments, the hydrogen is used to produce methanol 173. For example, methanol can be produced by directly hydrogenating pure carbon dioxide with hydrogen on Cu/ZnO-based catalysts. Alternatively, hydrogen can be used to produce methanol according to the following reactions:

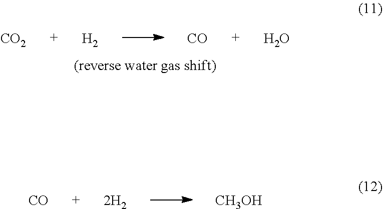

The methanol can be used as a fuel (e.g., mixed with gasoline) or can be used to produce a fuel (e.g., biodiesel).

In certain embodiments, the hydrogen is used to produce gasoline, diesel, and/or waxes using the Fischer-Tropsch process 174. The Fischer-Tropsch process refers to a collection of chemical reactions that converts syngas into liquid hydrocarbons, typically in the presence of metal catalysts under elevated pressures and temperatures. The Fischer-Tropsch process is well known. In the embodiments including a Fischer-Tropsch process, the hydrogen may be used to supplement another gas feed containing carbon monoxide and/or carbon dioxide in order to provide the required H₂:CO (e.g., about 2).

In certain embodiments, the hydrogen is used in the hydroprocessing (e.g., hydrocracking and/or hydrotreating) 175 of renewable fats and/or oils (e.g., algae, jatropha, tallows, camelina, pyrolysis oil produced from biomass, etc.) to produce, for example, gasoline, diesel, and/or jet fuel. Such embodiments are particularly advantageous as the renewable fuels can have reduced carbon intensity and/or be fully renewable.

In certain embodiments, the hydrogen is used in the hydroprocessing (e.g., hydrocracking and/or hydrotreating) 175 of crude-oil derived liquid hydrocarbon. For example, in certain embodiments the hydrogen (i.e., at least the renewable hydrogen) is incorporated into a crude-oil derived liquid hydrocarbon to produce, for example, gasoline, diesel, and/or jet fuel having renewable content (e.g., see U.S. Pat. Nos. 8,658,026, 8,753,854, 8,945,373, 9,040,271, 10,093,540, 10,421,663, and 10,723,621, 10,981,784). The term “crude oil derived liquid hydrocarbon”, as used herein, refers to any carbon-containing material obtained and/or derived from crude oil that is liquid at standard ambient temperature and pressure. The term “crude oil”, as used herein, refers to petroleum extracted from geological formations (e.g., in its unrefined form). Crude oil includes liquid, gaseous, and/or solid carbon-containing material from geological formations, including oil reservoirs, such as hydrocarbons found within rock formations, oil sands, or oil shale. The term “renewable content”, as used herein, refers to the portion of the fuel(s) that is recognized and/or qualifies as renewable (e.g., a biofuel) under applicable regulations. As will be understood by those skilled in the art, the quantification of the renewable content can be determined using any suitable method and is typically dependent upon the applicable regulations.

While producing a hydrogen product, such as a renewable hydrogen product, is advantageous, it is particularly advantageous when the hydrogen is used as feedstock for a production process (e.g., to produce a fuel, fuel intermediate, or chemical product). It can be particularly advantageous when the renewable hydrogen is used as feedstock for producing a transportation fuel. Using the renewable hydrogen in a production process can reduce GHG emissions associated with production process, and when the production process produces a fuel, can impart renewable content to the fuel and/or reduce the carbon intensity of the fuel. The GHG reductions can be significant, particularly when the renewable hydrogen has a negative carbon intensity. Advantageously, using renewable hydrogen in the hydroprocessing of crude-oil derived liquid hydrocarbon 175 can produce long-haul trucking fuel (e.g., diesel), shipping fuel (e.g., heavy fuel oil), aviation fuel (e.g., kerosene, jet fuel) and/or district heating fuel (e.g., heating oil). Such fuels can replace and/or be used to displace the corresponding petroleum based fuel (e.g., are drop-in fuels). Further advantageously, such fuels can be produced at existing oil refineries using existing equipment. In one embodiment, the renewable hydrogen is used in the hydroprocessing (e.g., hydrocracking and/or hydrotreating) of crude-oil derived liquid hydrocarbon to produce aviation fuel having renewable content. This embodiment is particularly advantageous as it could help decarbonize commercial air travel and/or extend the life of older aircraft types by lowering their carbon footprint.

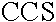

In general, carbon-containing material (e.g., derived from the biomass) can be stored using carbon capture and storage (CCS) 150. Carbon capture and storage (CCS) is a climate change mitigation technology that leads to a reduction in atmospheric carbon dioxide relative to the option of not using the technology. In general, CCS refers to one or more processes wherein carbon dioxide is captured from the atmosphere, or captured from a process that otherwise would release it to the atmosphere, and wherein the captured carbon is stored and/or used in a way that reduces the level of carbon dioxide in the atmosphere.

One example of CCS is where carbon dioxide is captured from an emitting source and then permanently stored underground. Another example of CCS is where carbon dioxide is captured and provided as a substitute to fossil-based carbon dioxide in an application that consumes fossil-derived carbon dioxide that is extracted or produced for the primary purpose of serving such application. In such an instance, the extraction or production is avoided, and the captured carbon dioxide that would otherwise be released does not enter the atmosphere, creating a reduction in atmospheric carbon dioxide levels relative to baseline of releasing the carbon dioxide. In managing the use of carbon dioxide to applications, distribution systems (e.g., pipelines) are often used to transport the carbon dioxide. One such use is in enhanced oil recovery (EOR) projects, where high-pressure carbon dioxide is injected into wells to carry more oil to the surface. Frequently, at least some of the carbon dioxide in the distribution system is fossil-based carbon dioxide obtained from naturally occurring underground carbon dioxide deposits. Injecting a quantity of captured carbon dioxide into such carbon dioxide distribution systems can prevent an equal quantity of carbon dioxide from being removed from the naturally occurring underground deposits, and result in a reduction atmospheric carbon dioxide levels by avoiding the release of such captured carbon dioxide.

For purposes herein, the phrase “carbon capture and storage” or “CCS” refers to carbon capture with substantially permanent storage (e.g., sequestration in geological formations) and/or carbon capture and use in beneficial applications (e.g., that consume carbon dioxide or use carbon dioxide to make a product), such that there is a reduction in atmospheric carbon dioxide relative to the absence of such carbon capture and storage and/or use. For purposes herein, providing carbon-containing material (e.g., gas such as carbon dioxide, liquid such as bio-oil, or solid such as biochar) as part of carbon capture and storage refers to providing the carbon-containing material for substantially permanent storage (e.g., sequestration in geological formations) and/or use in beneficial applications (e.g., that consume carbon dioxide or use carbon dioxide to make a product), such that there is a reduction in atmospheric carbon dioxide relative to the absence of carbon capture and storage and/or use. As will be understood by those skilled in the art, it can be advantageous for the carbon capture and storage technology to be selected such that it is recognized by the applicable regulatory authority for reducing lifecycle GHG emissions and/or mitigating climate change. For example, some regulations may require storage to have a maximum leakage rate (e.g., monitoring of carbon dioxide leakage from storage for a certain time period may be mandatory).

While CCS is often discussed in terms of directly capturing carbon dioxide using one or more carbon dioxide capture technologies such as adsorption, absorption, membrane, cryogenic, and/or chemical looping technologies, in some cases the carbon dioxide is captured from the atmosphere and converted to biomass via photosynthesis and at least part of the corresponding plants are used to produce bioenergy that makes biogenic carbon available for subsequent CCS. When such bioenergy production is integrated with CCS, this may be referred to as bioenergy with carbon capture and storage or BECCS. BECCS, which is a group of technologies that combine extracting bioenergy from biomass with CCS, has the potential to provide negative GHG emissions and thus may play an important role in commitments to reach net-zero carbon emissions. For example, in some cases, BECCS can be viewed as a process where biomass (e.g., plants) is used to capture carbon dioxide from the atmosphere, the biomass is processed to produce bioenergy (e.g., heat, electricity, fuels) while releasing carbon dioxide, and the carbon dioxide produced during the processing is captured and stored such that there is there is a net transfer of carbon dioxide from the atmosphere to storage. Alternatively, or additionally, carbon-containing material derived from the biomass (i.e., other than carbon dioxide) can be stored so as to prevent or delay such carbon from being released to the atmosphere (e.g., as methane and/or carbon dioxide).

In general, carbon capture and storage (CCS) in the instant disclosure includes storing and/or using carbon-containing material (e.g., at least partially derived from the biomass, and thus containing carbon captured from the atmosphere via photosynthesis) as part of one or more CCS processes. In general, the carbon-containing material can be provided as gas, liquid, and/or solid carbon-containing materials. In certain embodiments, the CCS also includes storing and/or using fossil-based carbon-containing material as part of one or more CCS processes (e.g., from hydrogen production). As will be understood by those skilled in the art, the carbon capture and storage technology used may be dependent on the type of carbon-containing material, the process, and/or applicable regulations (e.g., used to calculate lifecycle GHG emissions and/or qualify for fuel credits).

In certain embodiments, the CCS includes providing carbon dioxide produced from the process (e.g., produced from an ethanol fermentation process, produced from biomethane production process, produced from processing a residue of the biomethane production, and/or produced from hydrogen production) for storage and/or use as part of at least one carbon capture and storage process. In such embodiments, the carbon dioxide, which typically includes biogenic carbon dioxide (e.g., derived from the biomass), can also include fossil-derived carbon dioxide (e.g., if hydrogen production uses feed containing fossil-based methane-containing gas and biomethane). In general, the carbon dioxide can be captured using any suitable separation technology that can remove carbon dioxide from a gas mixture (e.g., biogas, syngas, flue gas). Alternatively, if the carbon dioxide is relatively pure, capturing the carbon dioxide can simply refer to collecting the carbon dioxide (e.g., in a pipe). It can be particularly advantageous to use gas separation techniques that provide a relatively pure carbon dioxide stream. Such techniques may for example, include vacuum PSA (VPSA), absorption processes (e.g., based on amines), and/or cryogenic separations (e.g., using temperatures below −10° C. or below −50° C.).

In certain embodiments, at least some of the carbon dioxide provided as part of the CCS is provided for storage (e.g., sequestration) in a subsurface formation (e.g., is trapped in geological formations, such as saline aquifers, oil and natural gas reservoirs, unmineable coal seams, organic-rich shales, or basalt formations). In certain embodiments, at least some of the carbon dioxide provided as part of CCS is provided for use in enhanced oil recovery (EOR). In certain embodiments, at least some of the carbon dioxide provided as part of CCS is provided for storage in a product (e.g., mineral sequestration). For example, carbon dioxide can react with metal oxides, such as magnesium and/or calcium oxides, to produce carbonates. Such mineral carbonates have many applications. Other suitable products may include building materials such as cement, concrete, or aggregates, chemicals, fuels, and/or food and beverages.

Carbon capture and storage of carbon dioxide, which is well-known in the art, may include one or more gas separation processes (e.g., used to separate the carbon dioxide from one or more other components of a gas mixture and/or to produce a stream of carbon dioxide that is of sufficient purity for storage, use, and/or transport). Carbon capture and storage of carbon dioxide often includes compression of the carbon dioxide and/or transport of the carbon dioxide.

Referring to FIGS. 1a, 1b, 2, 3, 4, and/or 5, carbon dioxide that is stored and/or used as part of the CCS can include carbon dioxide produced from biomethane production 120 and/or hydrogen production 160. In certain embodiments, a carbon capture and/or storage process may be integrated with and/or overlap another process. For example, the capture of carbon dioxide may correspond to one or more steps of the biomethane production (e.g., carbon dioxide scrubbing from methanation or biogas upgrading).

In certain embodiments, the CCS includes providing carbon dioxide produced from biomethane production for storage and/or use as part of at least one carbon capture and storage process. For example, in certain embodiments, the CCS includes providing carbon dioxide produced from anaerobic digestion as part of CCS. Such embodiments can be advantageous because some or all of the technologies used to upgrade the biogas produced by anaerobic digestion can also be used in the production of carbon dioxide suitable for storage and/or use.

In certain embodiments, the CCS includes providing carbon dioxide produced from hydrogen production for storage and/or use as part of at least one carbon capture and storage process. In general, carbon dioxide can be captured from any suitable part of the hydrogen production process. The hydrogen production processes in FIGS. 6a and 6b both emit carbon dioxide in the flue gas 12. In the older style unit illustrated in FIG. 6a, the carbon dioxide emitted in the flue gas is produced from the combustion of the fuel 3 used to fire the SMR furnaces. In the newer style unit illustrated in FIG. 6b, the carbon dioxide emitted in the flue gas is produced from both the methane reforming of feed 1 and the combustion of the fuel 3 used to fire the SMR furnace (e.g., off gas containing carbon dioxide is recycled). In certain embodiments, the carbon dioxide is captured from the syngas 25 (e.g., using vacuum pressure swing adsorption (VPSA) or an absorption amine unit). In certain embodiments, the carbon dioxide is captured from flue gas 12 (e.g., using an activated amine process). In certain embodiments, the carbon dioxide is captured from off gas 34. In certain embodiments, the carbon dioxide is captured from any combination of syngas, off gas, and flue gas. In general, it may be more technically and/or economically more feasible to capture the carbon dioxide from the syngas or off gas, as the flue gas may have a relatively low carbon dioxide concentration and/or may be at a lower pressure (e.g., atmospheric). In addition, the flue gas may contain nitrogen (e.g., CH₄/N₂ separations can be more challenging than CH₄/CO₂ separations).

In certain embodiments, the CCS includes providing carbon dioxide produced from processing a residue of the biomethane production process for storage and/or use as part of at least one carbon capture and storage process. In such embodiments, the processing can include any suitable processing, including for example, combustion, gasification, pyrolysis, and/or wet oxidation, while the residue can include any suitable material (e.g., typically liquid and/or solid) that is not converted to biogas or biomethane (e.g., digestate or biochar). In certain embodiments, the residue is waste or a byproduct of the biomethane production process. For example, referring to FIGS. 1a, 1b, 3, and 4, digestate 122a is processed, and carbon dioxide produced as a result of at least part of that processing is provided as part of CCS 150.

In certain embodiments, the CCS includes providing carbon dioxide produced by combusting at least a portion of the digestate for storage and/or use as part of at least one carbon capture and storage process. For example, in some embodiments, the digestate is subjected to a solids-liquid separation that provides a solids stream and a liquid stream. Such solids-liquid separation can be conducted using a screw press, centrifuge, etc. At least part of the solids stream is then combusted. Optionally, the solids are processed prior to combustion. For example, such processing can include washing, further drying (e.g., thermal drying), and/or compression (e.g., formed into bales, pellets, or brickettes). In certain embodiments, at least a portion of the liquids stream is alternatively and/or additionally combusted. For example, the liquid stream can be subjected to an evaporation process to produce relatively clean water that can be recycled back to the digester, thereby reducing water requirements while also reducing the amount of salts and/or trace metals (e.g., potassium, sodium, chromium, etc.) in the recycled water. This can increase biogas production (e.g., by removing inhibitors) and/or reduce lifecycle GHG emissions. The residue from evaporation may be provided for combustion.

Advantageously, the combustion of digestate can generate heat and/or power for the process (e.g., without requiring a substantial about of additional heat and/or power). For example, electricity can be produced by combusting at least part of the digestate in a boiler configured to produce high pressured steam for electricity generation. Optionally, at least part of digestate is combusted with another material, such as biomass from a different feedstock (e.g., wood chips). As will be understood by those skilled in the art, the combustion of digestate and/or the other material can produce a flue gas containing carbon dioxide, which can be captured and provided for storage and/or use as part of the carbon capture and storage to further reduce the lifecycle GHG emissions of the biomethane, renewable hydrogen produced from the biomethane, and/or fuel, fuel intermediate, or chemical produced form the biomethane and/or hydrogen. In addition, the use of the heat and/or power produced by the combustion can further reduce the lifecycle GHG emissions of the biomethane, renewable hydrogen produced from the biomethane, and/or fuel, fuel intermediate, or chemical produced form the biomethane and/or hydrogen. The combustion of at least the solids component of the digestate can be advantageous because it can contain a significant amount of lignin, the energy content of which otherwise would be wasted. In addition, the combustion of digestate may be advantageous over the combustion of raw biomass, as the upstream processing may result in fewer alkali salts (e.g., potassium salts) being present during the combustion (e.g., relative to combustion of raw biomass).

Further advantageously, the combustion of digestate can convert a material that may otherwise decompose and/or or be challenging to store as part of a carbon capture and storage process, into a material (e.g., carbon dioxide gas stream) that is compatible with established carbon capture and storage methods (e.g., geological storage of carbon dioxide).

In certain embodiments, the CCS includes providing carbon dioxide produced from a process that includes subjecting at least a portion of the digestate to gasification and/or pyrolysis for storage and/or use as part of at least one carbon capture and storage process. For example, in some embodiments, the digestate is subjected to a solids-liquid separation that provides a solids stream and a liquid stream. Such solids-liquid separation can be conducted using a screw press, centrifuge, etc. At least part of the solids stream is then subjected to gasification and/or pyrolysis. Optionally, the solids are processed prior to gasification and/or pyrolysis. For example, such processing can include washing, further drying (e.g., thermal drying), and/or compression (e.g., formed into bales, pellets, or brickettes). Advantageously, the gasification and/or pyrolysis of the digestate produces syngas that can be used in fuel cells to produce electricity for the process, or can be combusted to generate heat and/or power for the process. Further advantageously, the syngas contains carbon dioxide, which can be captured (e.g., pre- or post-combustion) and provided for storage and/or use as part of at least one carbon capture and storage process to further reduce the lifecycle GHG emissions of the biomethane, renewable hydrogen produced from the biomethane, and/or fuel, fuel intermediate, or chemical produced form the biomethane and/or hydrogen. Producing heat and/or power from the combustion of syngas can be advantageous over producing heat and/or power from the combustion of digestate, because such electric power can be generated in engines and/or gas turbines, which may be cheaper and more efficient that the steam cycle used in incineration, and that the carbon dioxide can be captured from the syngas (i.e., pre-combustion) rather than post-combustion. For example, electricity can be produced by combusting at least part of the syngas using Stirling-engine based combined heat and power (CHP) technology. In certain embodiments, carbon dioxide is captured pre-combustion, thereby enabling the capture of carbon dioxide from gas streams having relatively high carbon dioxide contents and/or pressures, while also providing a stream enriched in hydrogen for combustion and/or for use in one or more fuel cells. In certain embodiments, the gasification and/or pyrolysis of at least part of the digestate produces a residue (e.g., waste and/or byproduct), at least part of which is combusted, thereby producing carbon dioxide that can be provided as part of carbon capture and storage. For example, gasification and/or pyrolysis can produce biochar that can be combusted, while pyrolysis can also produce biooil that can be combusted. In certain embodiments, the digestate, or a stream derived therefrom, is processed with fossil fuels. For example, solid digestate may be gasified with coal, while pyrolysis oil may be converted to electrical power through co-combustion in a conventional fossil fuel power plant.

In certain embodiments, the CCS includes providing carbon dioxide produced from a process that includes subjecting at least a portion of the digestate to wet oxidation for storage and/or use as part of at least one carbon capture and storage process. Advantageously, such wet oxidation can produce carbon dioxide that can be captured and provided as part of carbon capture and storage to further reduce the lifecycle GHG emissions of the biomethane, renewable hydrogen produced from the biomethane, and/or fuel, fuel intermediate, or chemical produced form the biomethane and/or hydrogen.

In certain embodiments, the CCS includes providing carbon-containing material that is a residue of the biomethane production, or is derived from such residue, as part of carbon capture and storage. In certain embodiments, the carbon-containing material is not biodegradable under the storage conditions. In certain embodiments, the storage is selected such that if the carbon-containing material does degrade, that carbon dioxide released from the degradation is trapped.

In certain embodiments, the CCS includes providing carbon-containing material that is a residue of the biomethane production as part of carbon capture and storage. For example, referring to FIG. 5, the carbon-containing material can be, for example, biochar. Biochar, which can be produced from gasification and/or pyrolysis of the biomass, can be recycled within the gasification and/or pyrolysis processes (e.g., to provide additional fuel for the process). Alternatively, biochar, which is biologically unavailable, can be provided as a soil amendment where it can store the carbon in the soil for centuries. In certain embodiments of the disclosure, the CCS includes providing biochar as a soil amendment (e.g., instead of recycling it within the process), or includes subjecting a carbon-containing material derived from the biomass and not converted to bioenergy (e.g., a portion of the digestate) to gasification and/or pyrolysis, and providing the biochar produced therefrom for soil amendment or some other external use. Advantageously, such process may also produce additional bioenergy from the biomass (e.g., fuel and/or electricity). In certain embodiments, the heat and/or electricity generated from gasification and/or pyrolysis of a by-product is used within the process (e.g., in the biomethane production process) in order to keep the carbon intensity of the biomethane, renewable hydrogen, and/or fuel produced therefrom below a certain limit (e.g., below 20, 10 or 0 gCO₂e/MJ).

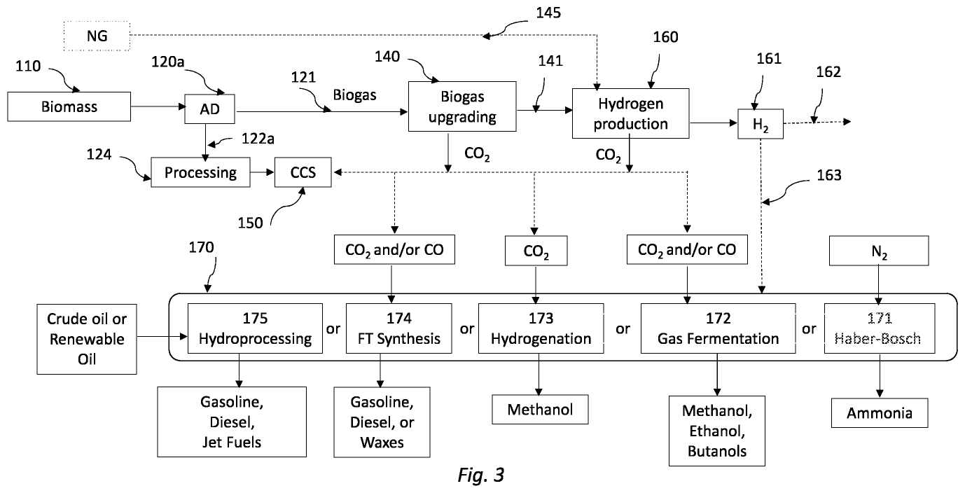

In certain embodiments, the CCS includes providing carbon-containing material produced from processing residue of the biomethane production (e.g., digestate) for storage and/or use as part of at least one carbon capture and storage process. Referring to FIGS. 1a, 1b, 3, or 4, the carbon-containing material can be, for example, biooil produced by pyrolysis or hydrothermal treatment of digestate 122a. In certain embodiments, CCS 150 includes providing liquid and/or solid carbon-containing material derived from residue of biomethane production (e.g., digestate) for storage and/or use as part of at least one carbon capture and storage process. In certain embodiments, CCS 150 includes providing carbon-containing material derived from residue of the biomethane production (e.g., digestate) for storage and/or use as part of at least one carbon capture and storage process. For example, bio-oil from hydrothermal liquefaction and/or pyrolysis oil can be sequestered in subsurface formations. Alternatively, such oils can be used as a renewable feedstock for chemicals and/or materials (e.g., plastics). For example, bio-oil from hydrothermal liquefaction and/or pyrolysis oil can be processed (e.g., hydroprocessed) to produce streams (e.g., naphtha), which can be used to produce plastic(s).

In certain embodiments, the CCS includes providing carbon-containing material derived from the biomass for storage and/or use as part of at least one carbon capture and storage process, where the carbon-containing material includes (i) carbon dioxide produced from the biomethane production process, (ii) carbon dioxide produced from the hydrogen production process, and (iii) carbon-containing material derived from part of the biomass not converted to biomethane (i.e., other than the carbon dioxide in (i)). In certain embodiments, CCS includes providing carbon-containing material derived from the biomass for storage and/or use as part of at least one carbon capture and storage process, where the carbon containing material includes (i) carbon dioxide produced from the biomethane production process, (ii) carbon dioxide produced from the hydrogen production process, and (iii) carbon-containing material derived from residue of the biomethane production process.

In certain embodiments, the CCS includes providing carbon-dioxide as part of carbon capture and storage, where the carbon dioxide includes (i) carbon dioxide produced from the biomethane production process (e.g., carbon dioxide from anaerobic digestion), (ii) carbon dioxide produced from the hydrogen production process (e.g., captured from syngas, off gas, and/or flue gas), and (iii) carbon dioxide produced from combusting at least part of a residue from the biomethane production process (e.g., digestate or biochar). In certain embodiments, CCS includes providing carbon-dioxide as part of carbon capture and storage, where the carbon dioxide includes (i) carbon dioxide produced from the biomethane production process (e.g., carbon dioxide from anaerobic digestion), and (ii) carbon dioxide produced from combusting at least part of a residue from the biomethane production process (e.g., digestate or biochar). In such embodiments, the carbon dioxide captured from each point can be processed and/or stored and/or used, together or separately.

Advantageously, providing carbon-containing material that is a residue, or is produced from processing a residue of the biomethane production process, can increase the amount of biogenic carbon from the biomass that can be provided for storage and/or use as part of at least one carbon capture and storage process. The resulting reduction in GHG emissions is significant when the captured carbon is derived from the digestate. Without being limiting in any way, and depending on the feedstock and/or process, about 50% of the carbon from the original biomass may end up in the biogas (e.g., as CO₂ and CH₄) while about 50% may end up in the digestate. Accordingly, providing carbon dioxide derived from the digestate (e.g., produced from combusting the digestate) as part of carbon capture and storage can significantly decrease the lifecycle GHG emissions of the biomethane, renewable hydrogen produced from the biomethane, and/or fuel, fuel intermediate, or chemical produced form the biomethane and/or hydrogen. Further advantageously, processing a residue of the biomethane production can facilitate the use the carbon from the biomass that otherwise would not be converted to bioenergy (e.g., heat, power, or biofuel, including, for example, biomethane, hydrogen, gasoline, diesel, jet fuel).

In certain embodiments, the CCS includes providing the carbon-containing material derived from the residue in one or more products. In this case, carbon-containing material derived from the biomass (e.g., and not converted to bioenergy) can be provided for storage and/or use as part of at least one carbon capture and storage process. For example, the carbon-containing material derived from the residue can be used to produce one or more products that makes the carbon unavailable for biodegradation (e.g., can be provided in products that provide continued sequestration benefits, such as building materials).

In certain embodiments, CCS includes sequestering a liquid and/or solid carbon-containing material derived from a part of the biomass not converted to bioenergy. Such materials can be sequestered indefinitely in a subsurface formation. For example, digestate can be subjected to a hydrothermal liquefaction to provide a bio-oil that can be sequestered. The pyrolysis of biomass, which can produce biomethane, can also produce pyrolysis oil, which can be sequestered. In some cases, the sequestration method is selected to prevent biodegradation of the material and/or trap GHGs in the event of biodegradation. In some cases, the material is treated in a process to reduce the potential for biodegradation. Sequestering a liquid carbon-containing material derived from the biomass may be advantageous in that injection into the storage area may be feasible and/or there may be fewer concerns related to leakage (i.e., relative to carbon dioxide sequestration).

The process(es) and/or system(s) of the instant disclosure produce at least one fuel (e.g., hydrogen, biomethane, ammonia), at least one fuel intermediate (e.g., hydrogen, biomethane, ammonia), and/or at least one product (e.g., chemical product, ammonia, fertilizer). The inclusion of CCS within various embodiments of the disclosure can reduce GHG emissions from the process (i.e., relative to no CCS) and/or reduce lifecycle GHG emissions of the product(s) of the process (i.e., relative to no CCS). In certain embodiments, combining CCS with fuel production provides a fuel that has a reduced carbon intensity (i.e., relative to with no CCS).

The term “carbon intensity” or “CI” refers to the quantity of lifecycle GHG emissions, per unit of fuel energy, and is often expressed in grams of CO₂ equivalent emissions per unit of fuel (e.g., gCO₂e/MJ or gCO₂e/MMBTU). As will be understood by those skilled in the art, lifecycle GHG emissions and/or carbon intensity are typically determined using Lifecycle Analysis (LCA), which identifies and estimates all GHG emissions in producing a fuel or product, from the growing or extraction of raw materials, to the production of the fuel or product, through to the end use (e.g., well-to-wheel). Those skilled in the art will understand that lifecycle GHG emissions and/or carbon intensity values for a given fuel or product can be dependent upon the methodology used (e.g., as required by the applicable regulatory authority).

In general, any methodology can be used to determine carbon intensity and/or lifecycle GHG emissions. However, when the fuel or product is specially treated for meeting a certain lifecycle GHG reduction threshold under certain regulations (e.g., is treated as clean or low carbon intensity hydrogen) and/or when the method includes obtaining one or more credits for the fuel or product and/or its production, the methodology will be selected to comply with the prevailing rules and regulations in the applicable jurisdiction (e.g., relevant to desired credits).

Methodologies for calculating carbon intensities and/or lifecycle GHG emissions according to various regulatory bodies are well known in the art and can be readily calculated by those of ordinary skill in the art. For example, in certain embodiments, the carbon intensities and/or lifecycle GHG emissions are determined using a LCA model, such as the GREET model. The GREET model, which is well-known by those skilled in the art, refers to “The Greenhouse gases, Regulated Emissions, and Energy use in Technologies Model” developed at Argonne National Laboratory (ANL) (e.g., grect.cs.anl.gov). In certain embodiments, the carbon intensities and/or lifecycle GHG emissions are determined based on the fuel/product being produced according to a certain pathway (e.g., a fuel pathway). For example, in certain embodiments, the carbon intensities are pathway certified carbon intensities or are regulatory default value carbon intensities. In general, the term “fuel pathway” refers to a collective set of processes, operations, parameters, conditions, locations, and technologies throughout all stages that the applicable agency considers appropriate to account for in the system boundary of a complete analysis of that fuel's lifecycle greenhouse gas emissions. In some cases, a fuel pathway can be a specific combination of three components, namely: (1) feedstock, (2) production process, and (3) product or fuel type. In certain embodiments, the carbon intensities are regulatory default value carbon intensities. For example, in the UK, biomethane produced from wet manure may have a default carbon intensity of 22 gCO2 eq/MJ when the digestate is fed to an open enclosure, and when the off-gas from biogas upgrading is not combusted, or may have a default carbon intensity of −100 gCO2 eq/MJ when the digestate is fed to closed enclosure, and when the off-gas from biogas upgrading is combusted. In certain embodiments, the carbon intensities (e.g., of biomethane feedstock) are determined using disaggregated default values (e.g., associated with certain feedstocks and/or steps in a supply chain) or a mixture of disaggregated default values and measured values (e.g., based on supply chain specific measured values). In certain embodiments, the carbon intensities (e.g., of biomethane feedstock) are determined (e.g., using a LCA) and then verified by the regulatory agency (e.g., the fuel pathway and/or corresponding carbon intensities can be approved by the regulatory agency) and/or by a verification body approved and/or appointed by the regulatory agency. The carbon intensity values recited herein are determined using the CA-GREET model (e.g., see, https://ww2.arb.ca.gov/resources/documents/lcfs-life-cycle-analysis-models-and-documentation), unless otherwise specified.

In certain embodiments, the CCS includes providing carbon-containing material (e.g., carbon dioxide) obtained and/or produced from more than one point in the process (e.g., multiple CCS processes). For example, such multi-tiered CCS can include providing carbon dioxide produced from multiple biogas plants for carbon capture and storage, wherein the biogas produced from such plants is used to provide the biomethane for hydrogen production, and/or can include various combinations of (a) storing carbon dioxide captured from biomethane production, (b) storing carbon dioxide captured from hydrogen production, and (c) storing gaseous, liquid and/or solid carbon-containing material derived from a part of the biomass not converted to biomethane (e.g., from the residue of biomethane production). Using carbon capture and storage, where the carbon is captured from multiple points in the process, decreases the amount of GHG emissions attributable to producing bioenergy from the biomass. In general, such carbon capture and storage can be achieved using one or more carbon capture and storage processes.

In certain embodiments, the CCS includes at least two CCS processes, including CCS of carbon dioxide from biomethane production (e.g., from biogas upgrading) and CCS of carbon dioxide from hydrogen production (e.g., captured from the syngas). In certain embodiments, the CCS includes at least three CCS processes, including CCS of carbon dioxide from biomethane production (e.g., from biogas from a purification process in gasification/methanation), CCS of carbon dioxide from hydrogen production (e.g., captured from the syngas, off gas, and/or flue gas), and CCS of carbon-containing material that is a residue biomethane production or is produced from a residue of biomethane production (e.g., biochar, a carbon-containing material derived from digestate, or a combination thereof). Advantageously, this three-tiered approach can significantly reduce the lifecycle GHG emissions of the fuel (e.g., hydrogen), fuel intermediate, or chemical product produced.

In certain embodiments, the CCS includes (a) storing carbon dioxide captured from biomethane production, and (b) storing residue or carbon-containing material derived from the residue (e.g., carbon dioxide from combusting at least some of the residue), but does not include storing carbon dioxide produced from hydrogen production.

Advantageously, the hydrogen produced from biomass according to certain embodiments of the instant disclosure can have lifecycle GHG emissions that are similar to and/or lower than green hydrogen (e.g., can be about net-zero, even when there is no carbon capture and storage of carbon dioxide from hydrogen production). In certain embodiments, the hydrogen production process can reduce GHG emissions (e.g., can be net negative). In certain embodiments, the carbon intensity of the fuel (e.g., renewable hydrogen or fuel produced using the renewable hydrogen) is negative and/or relatively low (e.g., below about 10 gCO₂e/MJ or below about 5 gCO₂e/MJ). In certain embodiments, the carbon intensity of the fuel (e.g., renewable hydrogen or fuel produced using the renewable hydrogen) is negative and/or relatively low (e.g., below about 10 gCO₂e/MJ or below about 5 gCO₂e/MJ) when there is no carbon capture and storage of carbon dioxide from hydrogen production. In certain embodiments, the carbon intensity of the fuel (e.g., renewable hydrogen or fuel produced using the renewable hydrogen) is negative and/or relatively low (e.g., below about 10 gCO₂e/MJ or below about 5 gCO₂e/MJ) when there is no carbon capture and storage of carbon dioxide from hydrogen production, and when carbon dioxide produced as part of biomethane production and carbon dioxide produced from the combustion of at least part of a residue from biomethane production is captured and stored. In certain embodiments, the type of biomass and/or quantity of carbon to be stored is selected such that the carbon intensity of the fuel is below a predetermined value (e.g., required for regulatory purposes). For example, in certain embodiments, the type of biomass is selected to keep the carbon intensity of the hydrogen below that of green hydrogen (e.g., below about 16 gCO₂e/MJ, below about 10 gCO₂e/MJ, or below zero).

In certain embodiments, hydrogen produced from the process has a carbon intensity lower than 0 gCO₂e/MJ, lower than −10 gCO₂e/MJ, lower than −20 gCO₂e/MJ, lower than −40 gCO₂e/MJ, or lower than −50 gCO₂e/MJ, of H₂, as calculated using the lower heating value (LHV). In certain embodiments, hydrogen produced from the process has a carbon intensity lower than about 0 kgCO₂e/kg H₂, lower than about 0.45 kgCO₂e/kg H₂, or lower than about 1.5 kgCO₂e/kg H₂.

While providing a zero carbon hydrogen is generally advantageous, it may be particularly advantageous if the carbon intensity is as low as possible when the hydrogen is used as a fuel or to produce a fuel, for fuel credit purposes.

In certain embodiments, the process includes generating, obtaining, or providing credits. Credits are used to incentivize renewable fuels, often in the transportation sector. For example, credits, such as fuel credits can be used to demonstrate compliance with some government initiative, standard, and/or program, where the goal is to reduce GHG emissions (e.g., reduce carbon intensity in transportation fuels as compared to some baseline level related to conventional petroleum fuels) and/or produce a certain amount of biofuel (e.g., produce a mandated volume or a certain percentage of biofuels). The target GHG reductions and/or target biofuel amounts may be set per year or for a given target date. Some non-limiting examples of such initiatives, standards, and/or programs include the Renewable Fuel Standard Program (RFS2) in the United States, the Renewable Energy Directive (RED II) in Europe, the Fuel Quality Directive in Europe, the Renewable Transport Fuel Obligation (RTFO) in the United Kingdom, and/or the Low Carbon Fuel Standards (LCFS) in California, Oregon, or British Columbia). Credits can also be used to incentivize other products associated with reduced carbon or greenhouse gas emissions, such as for example, producer or production credits for clean hydrogen or credits for products made using clean hydrogen.

The term “credit”, as used herein, refers to any rights or benefits relating to GHG or carbon reduction including but not limited to rights to credits, revenues, offsets, GHG gas rights, tax benefits, government payments or similar rights related or arising from emission reduction, trading, or any quantifiable benefits (including recognition, award or allocation of credits, allowances, permits or other tangible rights), whether created from or through a governmental authority, a private contract, or otherwise. A credit can be a certificate, record, serial number or guarantee, in any form, including electronic, which evidences production of a quantity of hydrogen or fuel meeting certain life cycle GHG emission reductions relative to a baseline (e.g., a gasoline baseline) set by a government authority. Credits for low CI hydrogen may be set by regulatory authority and provided in many forms, e.g., producer credits and the like. Non-limiting examples of fuel credits include RINs and LCFS credits. A Renewable Identification Number (or RIN), which is a certificate that acts as a tradable currency for managing compliance under the RFS2, may be generated for each gallon of biofuel (e.g., ethanol, biodiesel, etc.) produced. A Low Carbon Fuel Standard (LCFS) credit, which is a certificate which acts as a tradable currency for managing compliance under California's LCFS, may be generated for each metric ton (MT) of CO₂ reduced.

In general, the requirements for obtaining, generating, or causing the generation of credits can vary by country, the agency, and or the prevailing regulations in/under which the credit is generated. For example, in many cases, fuel credit generation may be dependent upon a compliance pathway (e.g., predetermined or applied for) and/or the biofuel meeting a predetermined GHG emission threshold. For example, with regard to the former, the RFS2 categorizes biofuel as cellulosic biofuel, advanced biofuel, renewable biofuel, and biomass-based diesel. With regard to the latter, to be a renewable biofuel under the RFS2, corn ethanol should have lifecycle GHG emissions at least 20% lower than an energy-equivalent quantity of gasoline (e.g., 20% lower than the 2005 EPA average gasoline baseline of 93.08 gCO₂c/MJ). In low carbon-related fuel standards, biofuels may be credited according to the carbon reductions of their pathway. For example, under California's LCFS, each biofuel is given a carbon intensity score indicating their GHG emissions as grams of CO₂ equivalent per megajoule (MJ) of fuel, and fuel credits are generated based on a comparison of their emissions reductions to a target or standard that may decrease each year (e.g., in 2019, ethanol was compared to the gasoline average carbon intensity of 93.23 gCO₂e/MJ), where lower carbon intensities generate proportionally more credits.

In certain embodiments, the process includes monitoring inputs and/or outputs from each of the biogas production, biomethane production, hydrogen production, and/or CCS. In this case, each of the inputs is a material input or energy input and each of the outputs is a material output or an energy output. Monitoring inputs and/or outputs of these process may facilitate calculating and/or verifying GHG emissions of the process, calculating and/or verifying carbon intensity of the fuel, fuel intermediate, or chemical product, may facilitate fuel credit generation (e.g., based on volumes of fuel produced), and/or may facilitate determining renewable content (e.g., when co-processing renewable and non-renewable fuels). Monitoring can be conducted over any time period (e.g., monthly statements, etc.). Monitoring can be conducted in conjunction with and/or using any suitable technology or combination of technologies that enables measurement of material and/or energy flows.

As described herein, certain embodiments of the instant disclosure relate to a hydrogen production process having a relatively low lifecycle GHG emissions (e.g., hydrogen having a carbon intensity that is low relative to green hydrogen and/or hydrogen produced by the SMR of fossil-based natural gas). For example, although producing biomethane (as opposed to raw biogas or cleaned biogas) adds an additional processing steps and/or cost, it may improve the process efficiency and/or carbon intensity while exploiting infrastructure used for transporting and/or processing natural gas. In addition, it may aid in monitoring inputs and/or outputs of some of the processes. Transporting the biomethane using a natural gas distribution system may also facilitate the use of biomethane having a relative low carbon intensity. For example, while biomethane from landfill gas may have a carbon intensity of about 40-50 gCO₂e/MJ, biomethane produced from manure is typically lower (e.g., dairy manure may have CI of about −270 gCO₂e/MJ, while swine manure may have a CI that is about −350 gCO₂e/MJ). Using biomethane having a carbon intensity that is less than 0 gCO₂e/MJ can significantly reduce the carbon dioxide of hydrogen produced therefrom. In certain embodiments, the biomethane is produced from manure livestock. In certain embodiments, the biomethane has a carbon intensity less than 0 gCO₂e/MJ, less than −10 gCO₂e/MJ, or less than −20 gCO₂e/MJ of CH₄. In addition, the carbon intensity of the hydrogen and/or fuel, fuel intermediate, or chemical product produced therefrom may have a reduced carbon intensity as a result of one or more CCS processes (i.e., relative to if there is no CCS).

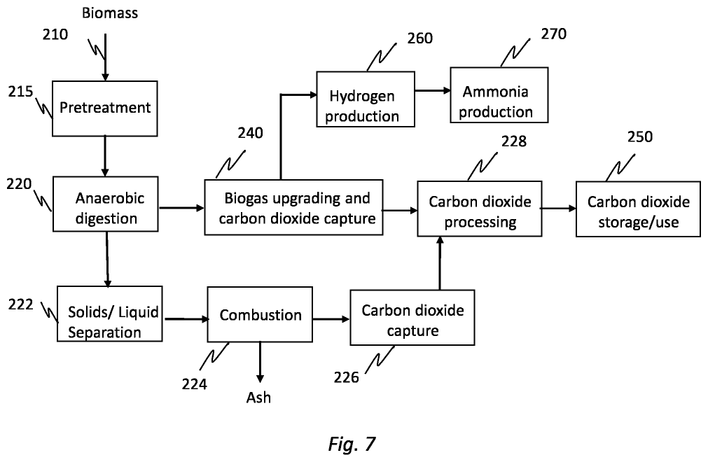

Referring to FIG. 7, there is shown an embodiment wherein biomass 210 is converted to ammonia. The biomass 210, which in this embodiment is fibrous biomass such as straw, is subjected to a pretreatment 215 prior to anaerobic digestion 220. The pretreatment includes subjecting the fibrous biomass to a size reduction, thereby reducing the average size of the fibrous biomass. The average particle size of the fibrous biomass can be measured by passing the fibrous biomass through screens having different mesh sizes (e.g., half inch, quarter inch, one eighth inch, etc.). For example, prior to size reduction very little straw from a bale will pass through a screen with a mesh size of one inch, but after chopping and/or grinding, a significant amount of the straw may pass through such screen.

The anaerobic digestion 220 produces biogas and digestate. In general, it can be advantageous to conduct the anaerobic digestion in the presence of added nutrients. The biogas, which contains at least methane and carbon dioxide, is subjected to biogas upgrading 240. The digestate is subjected to a solids/liquid separation 222, which produces a solids stream and a liquid stream. At least a portion of the solids stream is combusted 224 (e.g., after optional further drying and/or compression). At least a portion of the liquid stream is recycled back to the anerobic digestion (not shown), thereby reducing freshwater usage and/or liquid digestate storage requirements. For example, the liquid stream can be subjected to an evaporation step (not shown), which produces relatively clean water for recycling and a remaining component (e.g., containing salts and trace metals such as, for example, potassium, sodium, chromium, etc.). The remaining component can be combusted, disposed of, and/or processed to recover one or more of the components (not shown). For example, one or more compounds can be recovered prior to combustion with the solids stream 224. The combustion 224 produces heat and/or power that can be used in the process (e.g., displacing the use of fossil fuels). Since straw has a significant lignin content, and since a significant amount of the lignin is not typically digested, combusting at least the solids can allow for energy recovery from this lignin-rich fraction. In addition, the combustion 224 produces flue gas (e.g., from the boiler) containing carbon dioxide that is captured 226 and provided to carbon dioxide processing 228 prior to carbon dioxide storage/use 250. Accordingly, in addition to providing heat and/or power (e.g., for the process), the combustion converts at least part of the solid material to carbon dioxide, which advantageously can be processed and/or stored together with carbon dioxide from the biogas. This can simplify the process relative to CCS of different materials (e.g., gas with solid and/or liquid).

Biogas upgrading 240 produces biomethane, which is provided to hydrogen production 260. Biogas upgrading 240 also provides carbon dioxide (e.g., captured as part of biogas upgrading) that is provided for carbon dioxide processing 228. In general, the carbon dioxide captured from biogas upgrading 240 and/or from combustion 224 can be processed 228 and/or stored/used 250 together or separately. Carbon dioxide processing 228 typically includes dehydration, compression, chilling, and/or transport to a carbon capture and storage hub/site. Optionally, carbon dioxide processing includes producing liquid carbon dioxide (e.g., for transport to a carbon capture and storage hub/site). In this embodiment, the storage 250 includes sequestering the carbon dioxide in one or more suitable geological formations (not shown).

Hydrogen production 260 includes one or more methane reforming steps that converts feedstock to syngas. At least one of the methane reforming steps includes steam methane reforming, which also produces flue gas. The syngas is subjected to hydrogen purification, which produces a gas enriched in hydrogen and off gas. Carbon dioxide is captured from the syngas, from the off gas, and/or from the flue gas. Since the feedstock for methane reforming contains biomethane (e.g., where the biomethane is provided as feed for the reforming reactions and/or as fuel for producing heat for the reforming reactions), at least some of the captured carbon dioxide can be derived from the biomass (e.g., is biogenic).

The gas enriched in hydrogen is catalytically reacted with nitrogen to produce ammonia 270 (e.g., via the Haber-Bosch process).

Advantageously, this process facilitates the production of ammonia from a fibrous feedstock, such as straw. Since digested straw typically has a significant lignin content, the combustion of the digestate can provide a substantial amount of energy for the process. In addition, since the process includes multiple carbon capture processes, more carbon can be sequestered, which can facilitate obtaining fuel credits (e.g., hydrogen producer credits), which can increase economic feasibility.

Further advantageously, such processes can be preferable over processes that produce ammonia by gasifying raw biomass to produce syngas, which is used in ammonia production. For example, although gasifying raw biomass can obviate the size reduction, relatively long digestion times, and biogas upgrading typically associated with anaerobic digestion, and/or can be conducted with CCS from only one point (e.g., from the syngas, instead of from the biogas and from the digestate), the present disclosure can obviate one or more disadvantages of transporting the biomass to the gasification plant, thereby increasing the availability of biomass derived hydrogen for the ammonia production. For example, in this process, the biomass derived gas such as biomethane and/or hydrogen can be transported by pipeline, if required.

The terminology used herein is for the purpose of describing certain embodiments only and is not intended to be limiting of the invention. For example, as used herein, the singular forms “a,” “an,” and “the” may include plural references unless the context clearly dictates otherwise. The terms “comprises”, “comprising”, “including”, and/or “includes”, as used herein, are intended to mean “including but not limited to.” The term “and/or”, as used herein, is intended to refer to either or both of the elements so conjoined. The phrase “at least one” in reference to a list of one or more elements, is intended to refer to at least one element selected from any one or more of the elements in the list of elements, but not necessarily including at least one of each and every element specifically listed within the list of elements. Thus, as a non-limiting example, the phrase “at least one of A and B” may refer to at least one A with no B present, at least one B with no A present, or at least one A and at least one B in combination. In the context of describing the combining of components by the “addition” or “adding” of one component to another, or the separating of components by the “removal” or “removing” of one component from another, those skilled in the art will understand that the order of addition/removal is not critical (unless stated otherwise). The terms “remove”, “removing”, and “removal”, with reference to one or more impurities, contaminants, and/or constituents of biogas, includes partial removal. The terms “cause” or “causing”, as used herein, may include arranging or bringing about a specific result (e.g., a withdrawal of a gas), either directly or indirectly, or to play a role in a series of activities through commercial arrangements such as a written agreement, verbal agreement, or contract. The term “associated with”, as used herein with reference to two elements (e.g., a fuel credit associated with the transportation fuel), is intended to refer to the two elements being connected with each other, linked to each other, related in some way, dependent upon each other in some way, and/or in some relationship with each other. The terms “first”, “second”, etc., may be used to distinguish one element from another, and these elements should not be limited by these terms. The term “plurality”, as used herein, refers to two or more. The term “providing” as used herein with respect to an element, refers to directly or indirectly obtaining the element and/or making the element available for use. The terms “upstream” and “downstream”, as used herein, refer to the disposition of a step/stage in the process with respect to the disposition of other steps/stages of the process. For example, the term upstream can be used to describe a step/stage that occurs at an earlier point of the process, whereas the term downstream can be used to describe a step/stage that occurs later in the process. Unless defined otherwise, all technical and scientific terms used herein have the same meanings as commonly understood by one of ordinary skill in the art.

If course, the above embodiments have been provided as examples only. It will be appreciated by those of ordinary skill in the art that various modifications, alternate configurations, and/or equivalents will be employed without departing from the scope of the invention. Accordingly, the scope of the invention is therefore intended to be limited solely by the scope of the appended claims.

1-27. (canceled)

28. A method of producing fuel from biomass, the method comprising:
a) providing biomethane associated with a process comprising:
(i) subjecting the biomass to anaerobic digestion, thereby producing biogas and digestate, the biogas comprising methane and carbon dioxide;
(ii) upgrading at least some of the biogas, the upgrading comprising capturing at least some of the carbon dioxide produced in (i), at least some of the captured carbon dioxide provided for storage;
(iii) processing at least some of the digestate, the processing comprising combustion, gasification, pyrolysis, wet oxidation, or any combination thereof; and
(iv) capturing and storing at least some carbon dioxide generated from the digestate processed in (iii);
b) feeding the biomethane to methane reforming, the methane reforming producing syngas comprising carbon monoxide and hydrogen, the methane reforming further producing carbon dioxide, the carbon dioxide produced from the methane reforming being part of the syngas, part of a flue gas generated from the methane reforming, or a combination thereof;
c) capturing carbon dioxide from the syngas, from a stream derived from the syngas, from the flue gas, or any combination thereof, and providing at least some of the captured carbon dioxide for storage; and
d) feeding at least some of the hydrogen produced from the methane reforming to one or more processes that produce the fuel, wherein the fuel produced from the one or more processes has a carbon intensity that is dependent at least on the carbon dioxide captured in steps (ii), (iv) and (c) being stored.

29. The method according to claim 28, wherein the fuel comprises liquid hydrocarbon fuel, the liquid hydrocarbon fuel being gasoline, diesel, aviation fuel, shipping fuel, or any combination thereof.

30. The method according to claim 28, wherein the fuel comprises aviation fuel.

31. The method according to claim 29, wherein the one or more processes comprise at least one Fischer Tropsch process.

32. The method according to claim 29, wherein the one or more processes comprise reacting the hydrogen with crude oil derived liquid hydrocarbon.

33. The method according to claim 29, wherein the one or more processes comprise reacting the hydrogen with renewable fats, renewable oils, or a combination thereof.

34. The method according to claim 28, wherein the fuel comprises ammonia, ethanol, butanol, methanol, or any combination thereof.

35. The method according to claim 28, wherein the one or more processes comprise reacting at least some of the hydrogen produced from the methane reforming with at least some of the carbon monoxide produced from the methane reforming, at least some of the carbon dioxide produced from the methane reforming, or a combination thereof.

36. The method according to claim 28, wherein the processing comprises combustion, wet oxidation, or a combination thereof.

37. The method according to claim 28, wherein the fuel comprises hydrogen having a negative carbon intensity.

38. The method according to claim 29, wherein the fuel has a negative carbon intensity.

39. The method according to claim 38, wherein (iii) comprises combusting at least some of the digestate, and wherein (iv) comprises capturing and storing at least some carbon dioxide from flue gas generated from the combusting.

40. The method according to claim 28, wherein the biomass comprises agricultural residue.

41. The method according to claim 28, wherein (iii) comprises combusting at least some of the digestate, and wherein (iv) comprises capturing and storing at least some carbon dioxide from flue gas generated from the combusting.

42. The method according to claim 28, wherein (iii) comprises subjecting at least some of the digestate to the wet oxidation.

43. The method according to claim 28, wherein the methane reforming comprises steam methane reforming that produces the flue gas generated from the methane reforming, and wherein c) comprises capturing carbon dioxide from the flue gas generated from the methane reforming and does not include capturing carbon dioxide from the syngas.

44. The method according to claim 28, wherein at least some of the biomethane fed to methane reforming in b) is fuel used to produce heat for the methane reforming.

45. A method of producing fuel from biomass, the method comprising:
a) providing biomethane associated with a process comprising:
(i) subjecting the biomass to anaerobic digestion, thereby producing biogas and digestate, the biogas comprising methane and carbon dioxide;
(ii) upgrading at least some of the biogas, the upgrading comprising capturing at least some of the carbon dioxide produced in (i), at least some of the captured carbon dioxide provided for storage;
(iii) processing at least some of the digestate, the processing comprising combustion, wet oxidation, or a combination thereof; and
(iv) capturing and storing at least some carbon dioxide derived from the digestate processed in (iii);
b) feeding the biomethane to methane reforming, the methane reforming producing syngas comprising carbon monoxide and hydrogen, the methane reforming further producing carbon dioxide, the carbon dioxide produced from the methane reforming being part of the syngas, part of flue gas generated from the methane reforming, or a combination thereof; and
c) generating the fuel from one or more processes, the one or more processes fed at least some of the hydrogen generated from the methane reforming and fed:
(i′) at least some of the carbon dioxide generated from the methane reforming,
(ii′) at least some of the carbon monoxide generated from the methane reforming, or
(iii′) a combination of (i′) and (ii′),
wherein the fuel has a carbon intensity that is dependent at least on the carbon dioxide captured in steps (ii) and (iv) being stored.

46. The method of claim 45, wherein the fuel comprises liquid hydrocarbon fuel, and wherein the one or more processes comprises at least one Fischer Tropsch process.

47. A process for producing upgraded biogas for producing fuel, the process comprising:
subjecting biomass to anaerobic digestion, thereby producing biogas and digestate, the biogas comprising methane and carbon dioxide;
subjecting at least some of the biogas to biogas upgrading, thereby removing at least some of the carbon dioxide from the biogas and producing upgraded biogas;
providing at least some of the removed carbon dioxide as part of a first carbon capture and storage process;
processing at least part of the digestate, the processing comprising combustion, wet oxidation, or a combination thereof;
capturing carbon dioxide generated from the processing;
providing the captured carbon dioxide as part of the first carbon capture and storage process, a second other carbon capture and storage process, or a combination thereof; and
providing the upgraded biogas for producing the fuel.
